(19) United States
(12) Patent Application Publication
(10) Pub. No.: US 2026/0283455 A1
(43) Pub. Date: Sep. 24, 2026

(54) CLEANING ADAPTOR FOR AN ENDOSCOPE

(71) Applicant: MEDITECH ENDOSCOPY LTD, Chesterfield (GB)

(72) Inventor: Peter RAMSEY, Chesterfield (GB)

(21) Appl. No.: 19/472,915

(22) PCT Filed: Apr. 5, 2024
(86) PCT No.: PCT/GB2024/050918

§ 371 Date: Oct. 6, 2025

(30) Foreign Application Priority Data
Apr. 5, 2023 (GB) 2305104.8

(51) Int. Cl. A61B 1/12; A61B 1/00; A61B 1/015

(52) U.S. Cl. CPC A61B 1/125; A61B 1/00068; A61B 1/00137; A61B 1/015; A61B 1/126

(57) ABSTRACT

A cleaning adaptor for an air/water port of an endoscope is a unitary element forming a sealing plug. The sealing plug has a tubular stem extending between first and second ends, including a longitudinal passage. The longitudinal passage is open at the first end of said stem, A head portion closes the longitudinal passage at the second end of said stem and the head portion including a flange extending radially outwardly from the tubular stem. At least three annular seals extend radially outwardly from the tubular stem and are spaced apart along a length of the tubular stem. A transverse passage extends through the tubular stem and provides a fluid flow path between the longitudinal passage and an aperture in an outer surface of the tubular stem. The aperture is disposed between two of the at least three annular seals.

### FIELD OF THE INVENTION

This invention relates to a cleaning adaptor for engagement in an air-water port of a control section of an endoscope during a cleaning procedure. The cleaning adaptor is configured to replace an air-water valve of the endoscope to permit flushing of the air-water channel of the endoscope. This invention also relates to methods of using such a cleaning adaptor and to methods of flushing an air-water channel of an endoscope.

### BACKGROUND TO THE INVENTION

Endoscopes are now widely used in a number of medical procedures. An endoscope typically includes a light source connector, a control section, an umbilical cord connecting the light source connector to the control section, and an insertion tube terminating at a distal tip. The light source connector provides connections to a light source, an air pump, and a water bottle.

During use of the endoscope, an operator is able to use an air/water valve in the control section to control the delivery of air (from the air pump) or water (from the water bottle) to the distal tip through an air/water channel of the endoscope. A small metal nozzle is generally disposed at the distal tip of the endoscope to deflect air or water from the air/water channel across an image lens of the endoscope.

It will be appreciated that the air/water channel is necessarily of a small diameter. Furthermore, the air/water channel is actually formed by the two separate small diameter channels that only combine into a single channel proximate the distal tip of the endoscope.

After use of an endoscope it is essential that the endoscope is properly cleaned and sterilised before it is used again. Cleaning refers to the removal of visible debris, which may include both inorganic and organic substances. Cleaning usually involves a mechanical process, together with water and detergent. Following cleaning, sterilisation of the endoscope destroys microorganisms present on or in the endoscope. Sterilisation processes usually involve steam or a chemical sterilant. Following sterilisation, the endoscope undergoes high-level disinfection.

The presence of the metal nozzle at the distal tip means that it is not possible to brush the air/water channel. This, together with the small diameter of the air/water channel, as well as other factors, makes it difficult to clean and decontaminate the air/water channel.

Additionally, the air/water nozzle is the point of smallest internal diameter of the air/water channel system such that it can easily become blocked or obstructed by debris. During an endoscopic procedure, air infused into the organ being examined can force debris to back up into the nozzle and into the air/water channels. This debris can travel some distance back into the channels.

It is currently recommended that a preliminary cleaning routine is undertaken immediately after use of the endoscope and before the light source connector is detached from the light source/video processor. Current pre-clean practice is to flush air and water through the air/water channel. It is also recommended that the air and water channels (and any auxiliary channel) are also irrigated with detergent, not only to expel any blood, mucus and other debris, but also to check for blockages. It has been found, however, that routine pre-cleaning procedures for endoscopes do not remove biofilm reliably from endoscope channels. This leads to failures in the subsequent decontamination processes.

To enable this pre-clean or flushing of the air/water channel it is necessary to replace the air/water valve that is used during the procedure with a dedicated cleaning valve or adaptor. These cleaning valves allow a user to direct a flow of air or water (or detergent solution) down each of the separate smaller channels of the air/water channel.

Some of these cleaning valves are reusable and must, therefore, be designed to be thoroughly cleaned and sterilised after use to avoid cross-contamination between endoscopes. There is therefore significant cost involved in providing a suitable reusable cleaning valve.

To overcome this problem, a number of disposable or single-use cleaning valves have been developed. These disposable valves are, however, still relatively expensive to manufacture due to the complex arrangement of seals and passageways that must be provided to control the flow of air and water during the pre-clean procedure.

It is an object of the present invention to provide an improved cleaning adaptor for use in pre-clean procedures for flushing the air/water channel of an endoscope. In particular it is an object of the present invention to provide an improved cleaning adaptor that is cheaper and easier to manufacture, and is easier to use during the pre-clean procedure.

### SUMMARY OF THE INVENTION

A first aspect of the invention provides a cleaning adaptor for an air/water port of an endoscope, the cleaning adaptor being a unitary element comprising a sealing plug having:
a tubular stem extending between first and second ends and including a longitudinal passage, the longitudinal passage being open at the first end of said stem;
a head portion closing the longitudinal passage at the second end of said stem and the head portion including a flange extending radially outwardly from the tubular stem;
at least three annular seals extending radially outwardly from the tubular stem and being spaced apart along a length of the tubular stem; and
a transverse passage extending through the tubular stem and providing a fluid flow path between the longitudinal passage and an aperture in an outer surface of the tubular stem, the aperture being disposed between two of the at least three annular seals.

Preferably a first seal of the at least three annular seals is disposed at or proximate the first end of the stem, a second seal and a third seal of the at least three annular seals are disposed between the first seal and the second end of the stem, and the aperture is disposed between the second and third seals.

The cleaning adaptor may further comprise a weakened portion proximate the second end of the stem, the weakened portion being configured to rupture when a predetermined tensile force is applied to the weakened portion. The weakened portion may be disposed between the head portion and the annular seals. In some embodiments the weakened portion may comprise a notch or an indent in an external surface of the tubular stem.

The transverse passage preferably extends fully across the tubular stem and terminates at a first aperture on a first side of the stem and a second aperture on an opposite, second side of the stem.

In some embodiments the cleaning adaptor may further comprise a sealing cap connected to the sealing plug, the sealing cap comprising a plug portion for insertion into a suction cylinder of an endoscope and a cover portion including a sealing surface for contacting a collar surrounding an opening of said suction cylinder. The sealing cap may further comprise an annular retainer including an annular groove configured to engage with said collar surrounding said opening of said suction cylinder. In some embodiments the sealing plug may be connected to the sealing cap by a flexible connecting portion extending between the head portion and the cover portion.

In preferred embodiments the cleaning adaptor further comprises a grip element protruding from the head portion configured to be gripped by a user of the cleaning adaptor. In other embodiments the cleaning adaptor may further comprise a grip element protruding from the cover portion configured to be gripped by a user of the cleaning adaptor.

In preferred embodiments the cleaning adaptor is made from a resilient polymeric material.

A second aspect of the invention provides an assembly comprising:
an endoscope having an air/water port connected to air and water channels and including an air/water cylinder having an air inlet, an air outlet, a water inlet and a water outlet; and
a cleaning adaptor according to the first aspect of the invention, the sealing plug of the cleaning adaptor being disposed in the air/water cylinder such that the water inlet, air outlet and water outlet are in fluid communication with the longitudinal passage so that fluid is able to flow from the water inlet to both the air outlet and the water outlet, and at least two of the annular seals block fluid flow between the air inlet and any of the water inlet, the air outlet and the water outlet.

In preferred embodiments of the assembly the first end of the stem and a first one of the annular seals is disposed between the air inlet and the water inlet and between the air outlet and the water outlet; the air inlet is disposed between said first annular seal and a second one of the annular seals; and the air outlet and the aperture is disposed between said second annular seal and a third one of the annular seals.

Preferably the endoscope further comprises a suction port including a suction cylinder having an opening surrounded by a collar, and the cleaning adaptor comprises a sealing cap connected to the sealing plug, the sealing cap comprising a plug portion for insertion into a suction cylinder of an endoscope and a cover portion including a sealing surface for contacting a collar surrounding an opening of said suction cylinder. The sealing cap is engaged with the suction port such that the sealing surface of the cover portion is in contact with the collar to seal the suction cylinder.

A third aspect of the invention provides a method of cleaning air and water channels of an endoscope using a cleaning adaptor according to the first aspect of the invention, the endoscope having an air/water port connected to the air and water channels and including an air/water cylinder having an air inlet, an air outlet, a water inlet and a water outlet, and the method comprising:
inserting a sealing plug of the cleaning adaptor into the air/water port so that the tubular stem extends into the air/water cylinder and the air inlet is disposed between two annular seals such that there is no path for fluid flow between the air inlet and any of the water inlet, air outlet and water outlet, and the water inlet, air outlet and water outlet are in fluid communication with the longitudinal passage; and
providing a flow of liquid to the air/water cylinder through the water inlet, the longitudinal passage and the transverse passage forming a fluid flow path allowing the liquid entering the air/water cylinder to flow out of both the air outlet and the water outlet simultaneously.

Preferably the first end of the stem and a first one of the annular seals is disposed between the air inlet and the water inlet and between the air outlet and the water outlet, the air inlet is disposed between said first annular seal and a second one of the annular seals, and the air outlet and the aperture is disposed between said second annular seal and a third one of the annular seals.

Preferably the endoscope comprises an air channel in fluid communication with both the air inlet and a container holding a volume of liquid, and a water channel in fluid communication with the water inlet and the container, and the method comprises providing a flow of air in the air channel to pressurise the container and force the liquid in the container to flow through the water channel to the water inlet to provide the flow of liquid to the air/water cylinder.

The liquid may be water or a detergent solution.

In some embodiments the endoscope has a suction port connected to a suction channel, and the cleaning adaptor comprises a sealing cap connected to the sealing plug, the sealing cap comprising a plug portion for insertion into a suction cylinder of an endoscope and a cover portion including a sealing surface for contacting a collar surrounding an opening of said suction cylinder. In these embodiments the method of cleaning air and water channels of the endoscope preferably further comprises engaging the sealing cap with a collar around an opening of the suction port to seal the suction port and flushing water or a detergent solution through the suction channel.

In some embodiments the cleaning adaptor includes a grip element protruding from the head portion configured to be gripped by a user of the cleaning adaptor, and the method of cleaning air and water channels of the endoscope preferably comprises gripping the grip element and applying a force to the cleaning adaptor to pull the sealing plug out of the air/water cylinder. In other embodiments the cleaning adaptor includes a grip element protruding from the cover portion configured to be gripped by a user of the cleaning adaptor, and the method of cleaning air and water channels of the endoscope preferably comprises gripping the grip element and applying a force to the cleaning adaptor to disengage the sealing cap from the collar of the suction port and to pull the sealing plug out of the air/water cylinder. In preferred embodiments the cleaning adaptor comprises a weakened portion proximate the second end of the stem, and the method comprises breaking or rupturing the weakened portion by applying a tensile force to the weakened portion while applying a force to the cleaning adaptor to pull the sealing plug out of the air/water cylinder.

Preferred and/or optional features of each aspect and embodiment described above may also be used, alone or in appropriate combination, in the other aspects and embodiments also.

### BRIEF DESCRIPTION OF THE DRAWINGS

The invention will now be further described by way of example only and with reference to the accompanying drawings, in which like reference signs are used for like features, and in which:

FIG. 1 illustrates the internal fluid channels of a known endoscope;

FIG. 2 illustrates an air/water port and a suction port of a control section of a known

FIG. 3 illustrates a prior-art air/water valve seated in the air/water port of FIG. 2, the air/water valve being in a first position permitting a flow of air through the air/water port;

FIG. 4 illustrates the air/water valve of FIG. 3 in a second position permitting a flow of liquid through the air/water port;

FIG. 5 is a perspective view of a cleaning adaptor according to a first preferred embodiment of the invention, showing an underside or insertion side of the adaptor;

FIG. 6 is a perspective view of the cleaning adaptor of FIG. 5 showing an upper or cap portion of the adaptor;

FIG. 7 is a side view of the cleaning adaptor of FIG. 5;

FIG. 8 is a cross-sectional view of the cleaning adaptor of FIG. 5, along the line VIII-VIII of FIG. 11;

FIG. 9 is a end view of the cleaning adaptor of FIG. 5;

FIG. 10 is a sectional view of the cleaning adaptor of FIG. 5, along the line X-X of FIG. 11;

FIG. 11 is a top view of the cleaning adaptor of FIG. 5;

FIG. 12 is a view from an underside or insertion side of the cleaning adaptor of FIG. 5;

FIG. 13 is a sectional view of the cleaning adaptor of FIG. 5, along the line XIII-XIII of FIG. 7;

FIG. 14 illustrates the cleaning adaptor of FIG. 5 engaged with the air/water port and suction port illustrated in FIG. 2;

FIG. 15 is a perspective view of a cleaning adaptor according to a second preferred embodiment of the invention;

FIG. 16 is a further perspective view of the cleaning adaptor of FIG. 15;

FIG. 17 is a cross-sectional view of the cleaning adaptor of FIG. 15; and

FIG. 18 is a cross-sectional view of the cleaning adaptor of FIG. 15 engaged with an air/water port and a suction port of an endoscope, with the air/water channels and suction channels not shown for clarity.

### DESCRIPTION OF THE PREFERRED EMBODIMENTS

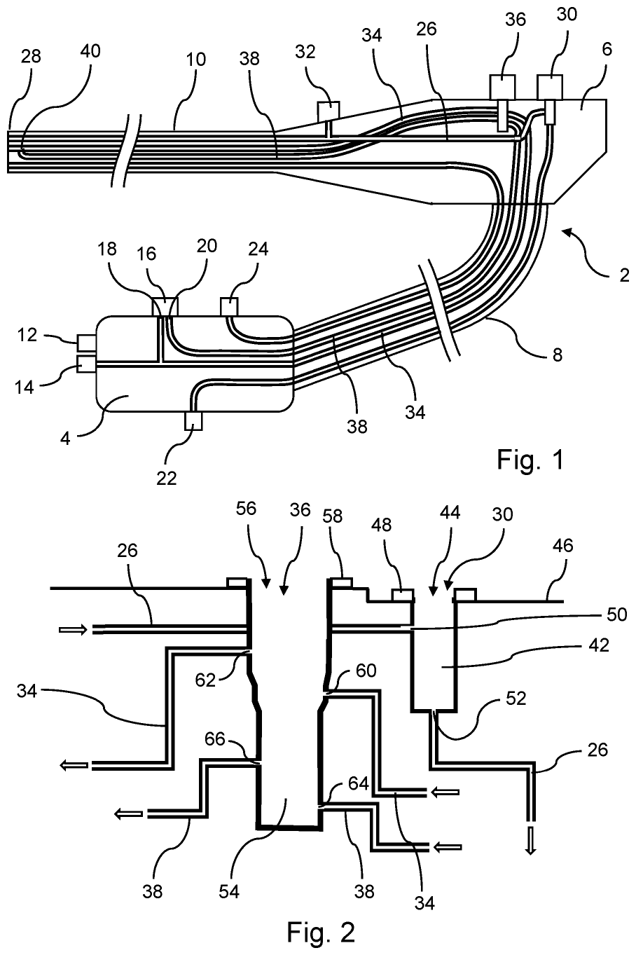

FIGS. 1 to 4 illustrate a known air/water system of an endoscope 2. The endoscope 2 includes a light source connector 4, a control section 6, an umbilical cord 8 connecting the light source connector 4 to the control section 6, and an insertion tube 10. The light source connector 4 comprises a connector 12 for connection to a light source and a connector 14 for connection to an air pump.

The light source connector 4 also includes a connector 16 for attachment of a water bottle. This water bottle connector 16 includes an air port 18 and a water port 20. The light source connector 4 further includes a connection 22 to a suction pump and a connection 24 to a water jet.

The endoscope 2 includes a suction/biopsy channel 26 that extends from the connection 22 to the suction pump to a distal end or tip 28 of the insertion tube 10. A first or proximal section of the suction/biopsy channel 26 extends from the light source connector 4 to a suction valve housed within a suction port 30 of the control section 6. A second or distal section of the suction/biopsy channel 26 extends from the suction port 30 to the tip 28 of the insertion tube 10. A branch off the second section of the suction/biopsy channel 26 terminates at a biopsy valve 32.

An air channel 34 of the endoscope extends from the connector 14 for connection to an air pump to the distal tip 28 of the insertion tube 10. A first section of the air channel 34 extends from the light source connector 4 to an air/water valve 35 housed within an air/water port 36 of the control section 6. A second section of the air channel 34 extends from the air/water port 36 to the tip 28 of the insertion tube 10.

Within the light source connector 4, a branch extends from the first section of the air channel 34 to the air port 18 of the water bottle connector 16 to permit a flow of air from the air channel 34 to a water bottle connected to the connector 16.

A water channel 38 of the endoscope extends from the water port 20 of the water bottle connector 16 to the distal tip 28 of the insertion tube 10. A first section of the water channel 38 extends from the light source connector 4 to the air/water valve housed within the air/water port 36 of the control section 6. A second section of the water channel 38 extends from the air/water port 36 to the tip 28 of the insertion tube 10.

Proximate the distal tip 28 of the insertion tube 10 the air and water channels 34, 38 combine into a single channel. Typically the air and water channels 34, 38 are referred to collectively as the air/water channel 40 of the endoscope 2.

Referring now to FIG. 2, the suction port 30 comprises a cylindrical recess or cavity 42 (referred to as a suction cylinder) for receiving the suction valve. The suction cylinder 42 creates an opening 44 in a surface 46 of the control section 6 that is, in this example, surrounded by a raised collar 48. The first section of the suction/biopsy channel 26 enters the suction cylinder 42 at a suction inlet 50 and the second section of the suction/biopsy channel 26 extends from a suction outlet 52 of the suction cylinder 42.

The air/water port 36 comprises a cylindrical recess or cavity 54 (referred to as an air/water cylinder) for receiving the air/water valve 35. The air/water cylinder 54 creates an opening 56 in the surface 46 of the control section 6 that is surrounded, in this example, by a raised collar 58. The openings 44, 56 of the suction port 30 and air/water port 36 are disposed adjacent each other in the control section 6. The first section of the air channel 34 enters the air/water cylinder 54 at an air inlet 60 and the second section of the air channel 34 extends from an air outlet 62 of the air/water cylinder 54. The first section of the water channel 38 enters the air/water cylinder 54 at a water inlet 64 and the second section of the water channel 38 extends from a water outlet 66 of the air/water cylinder 54.

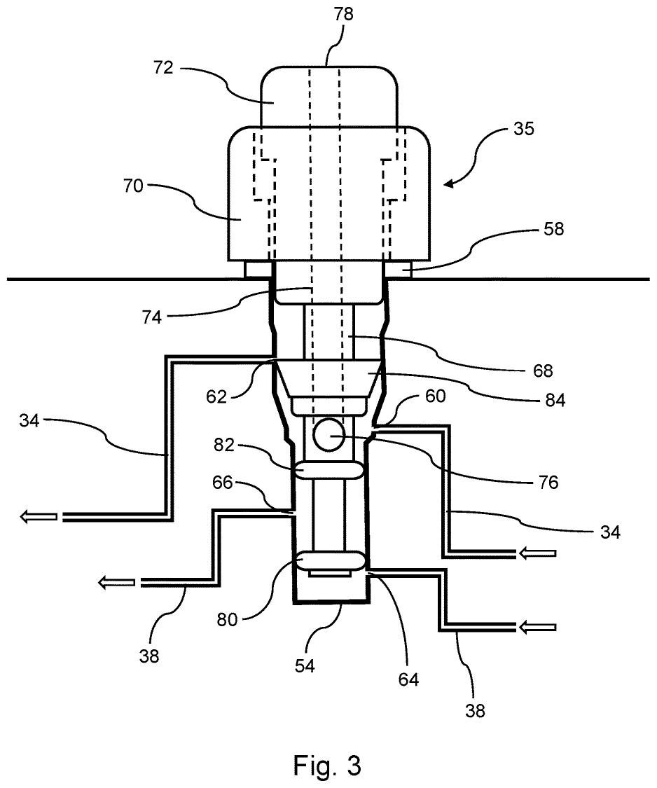

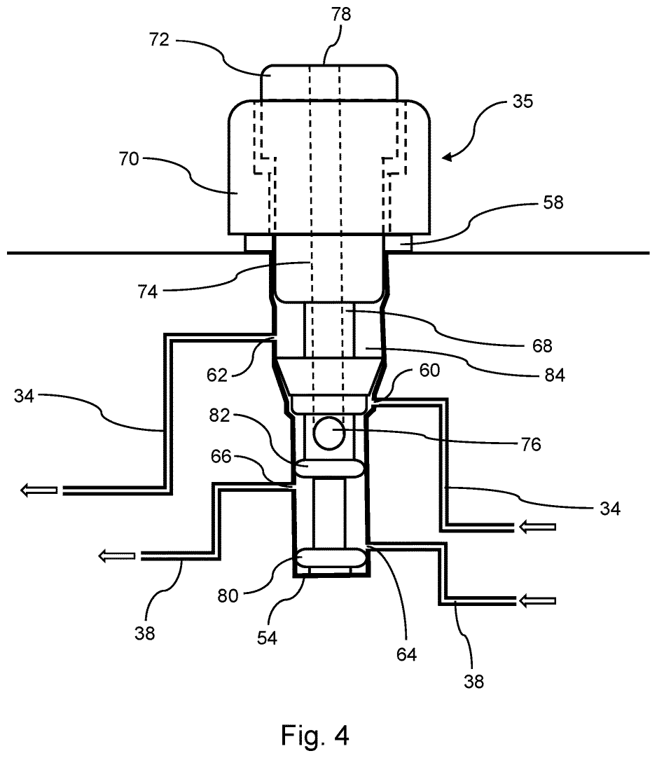

Referring now to FIGS. 3 and 4, a typical air/water valve 35 comprises a central stem 68 engaged with an attachment sleeve 70. The attachment sleeve 70 is configured to engage with the collar 58 to retain the valve 35 in the air/water cylinder 54. The stem 68 is engaged with the sleeve 70 so that the stem is movable between first and second positions. Typically the stem will be biased into the first position. A button portion 72 at a first or upper end of the stem 68 protrudes from the sleeve 70 and permits a user to manually move the stem from the first position to the second position.

The stem 68 includes a central passage 74 having an inlet aperture 76 and an outlet aperture 78. The outlet aperture 78 is provided in an upper surface of the button portion 72.

A plurality of seals extend circumferentially around the stem 68 spaced apart along a length of the stem 68. A first seal 80 is disposed at or proximate a second or lower end of the stem 68. A second seal 82 is disposed above the first seal 80 (further from the second end of the stem 68) between the inlet aperture 76 and the first seal 80. A third seal 84 is disposed above the second seal 82, between the inlet aperture 76 and the button portion 72. The seals 80, 82, 84 are configured such that they contact and seal against a circumferential wall of the air/water cylinder 54.

During use of the endoscope 2, a flow of air is constantly provided by the air pump. The air travels along the first section of the air channel 34 and enters the air port 18 at the air inlet 60. Air also travels along the branch extending from the first section of the air channel 34 into the water bottle connected to the connector 16. This air flow pressurises the water bottle and forces liquid (water) out of the water bottle and into the first section of the water channel 38.

With the stem 68 of the air/water valve in the first position (shown in FIG. 3), the first seal 80 prevents a flow of liquid from the water inlet 64 to the water outlet 66, such that no liquid (for example water) is able to flow along the second section of the water channel 38 to the distal tip 28. Air entering the air port 18 at the air inlet 60 is prevented from flowing out of the air outlet 62 by the third seal 84. The air flows through the inlet aperture 76 in the stem 68, through the central passage 74 and out of the outlet aperture 78.

When an operator wishes to direct air flow to the distal tip 28, a thumb or finger is used to gently cover the outlet aperture 78 with the stem 68 remaining in the first position. As air is no longer able to flow along the central passage 74 and out of the outlet aperture 78, the pressure between the second and third seals 82, 84 increases. At a defined pressure the third seal 84 is configured to deflect so permit a flow of air to the air outlet 62. In this way, air is able to flow along the second section of the air channel 34 to the distal tip 28.

When an operator wishes to direct a flow of water to the distal tip 28, pressure is applied to the button portion 72 of the valve 35 to move the stem 68 into the second position (shown in FIG. 4). In this second position the first seal 80 is no longer disposed between the water inlet 64 and water outlet 66 so that water is able to flow from the water inlet 64 to the water outlet 66 between the first and second seals 80, 82. The third seal 84 moves into a position to fully block the air inlet 60, such that the third seal 84 does not deflect and air flow to the air outlet 62 is prevented.

At all times, whether the stem 68 of the valve 35 is in the first or second position, the second seal 82 blocks fluid flow between the air inlet 60 and the water outlet 66 and between the water inlet 64 and the air outlet 62.

Following use of the endoscope 2, the air/water valve 35 is removed and replaced by a cleaning adaptor before pre-clean of the endoscope. A known cleaning adaptor is similar in construction to the air/water valve 35 described above but does not include a central passage that terminates at an outlet in the button portion. Instead a passage in a stem of the cleaning adaptor allows air to flow from the air inlet of the air/water port to both the air outlet and the water outlet concurrently when the cleaning adaptor is in a first position. When the button portion of the cleaning adaptor is depressed, the air inlet is blocked and passages in the stem portion allow liquid to flow from the water inlet to the air outlet and water outlet to allow flushing of the second section of each of the air and water channels.

Known cleaning adaptors are movable between first and second positions to enable the air/water channel to be flushed with both liquid (water or detergent solution) and air.

It has been found, however, that the efficacy of subsequent decontamination processes (sterilisation and disinfection), may be improved if the internal channels of the endoscope remain moist or wet. In particular, this may reduce biofilm adhesion within the channels leading to easier removal of the biofilm during cleaning, sterilisation and disinfection.

The present invention therefore provides a cleaning adaptor for an air/water cylinder of an endoscope that may be utilised in a pre-clean process to flush the air/water channel of the endoscope with water or a detergent solution.

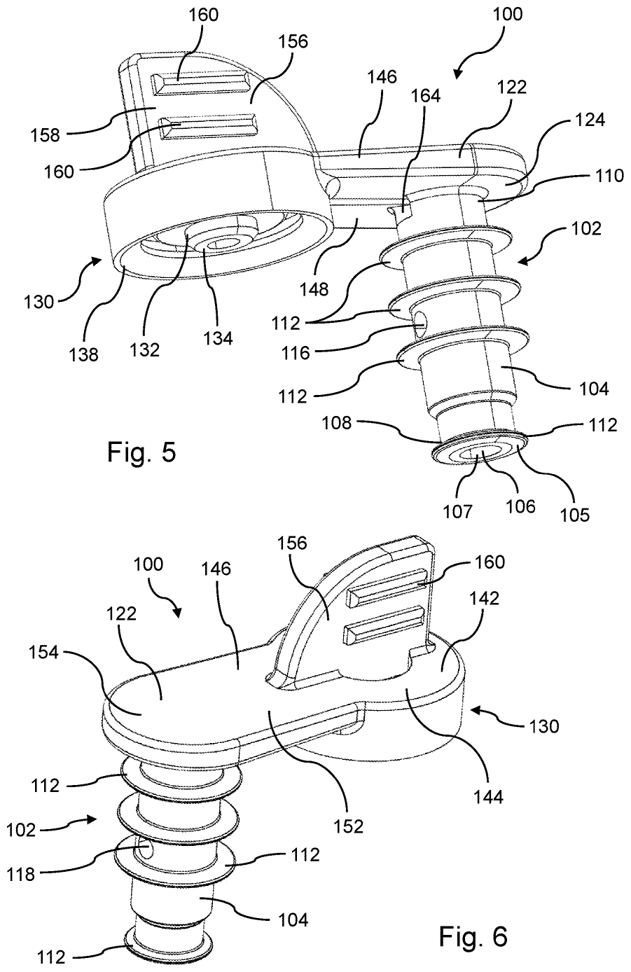

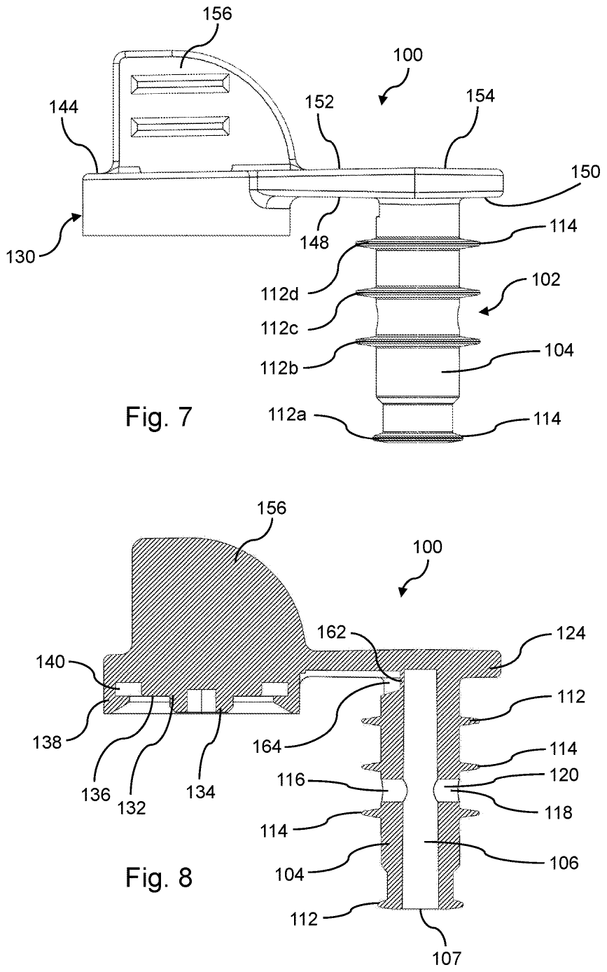

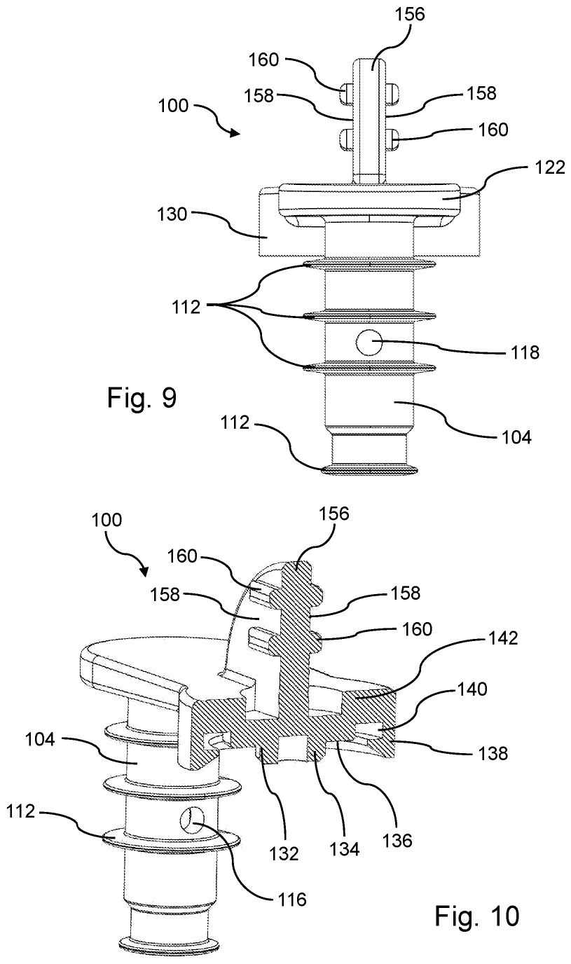

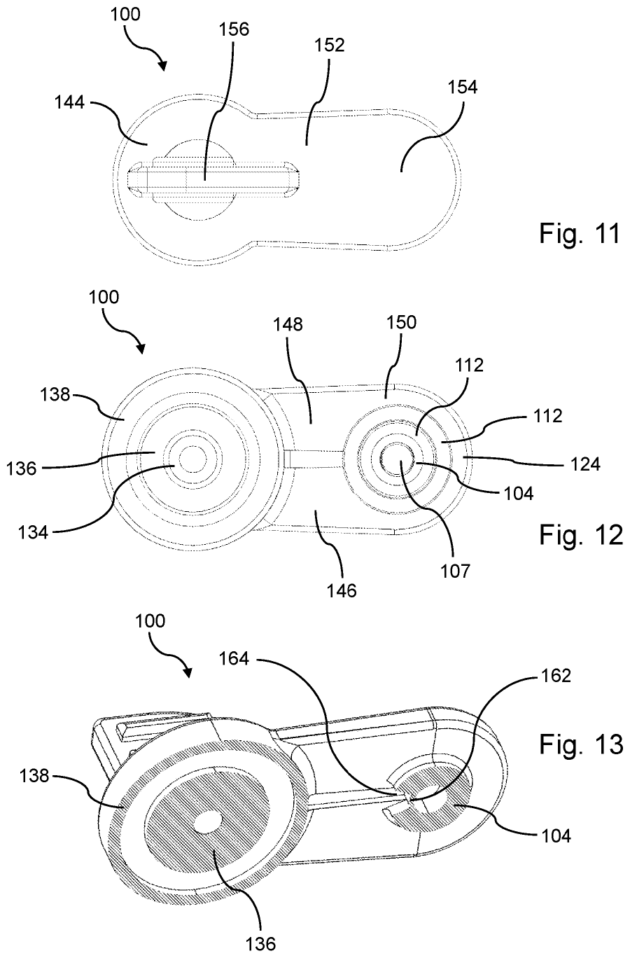

FIGS. 5 to 13 show a cleaning adaptor 100 according to a first preferred embodiment of the present invention. The cleaning adaptor 100 is a unitary, one-piece element. The cleaning adaptor 100 has no moving parts other than due to the inherent flexibility of some parts of the adaptor 100 as described further below. The cleaning adaptor is preferably made from a suitable polymeric material, having a degree of elasticity or flexibility. The cleaning adaptor may be made from silicone. The cleaning adaptor is preferably made from a single material. The cleaning adaptor may be injection moulded.

The cleaning adaptor 100 comprises a sealing plug 102 for insertion into the air/water cylinder of the air/water port of the endoscope. The sealing plug 102 comprises an elongate tubular stem 104 having a passage or bore 106. The stem 104 extends between a first, lower or distal end 108 and a second, upper end 110. The passage 106 extends longitudinally through the stem 104 between the first and second ends 108, 110. An opening 107 at the end of the passage 106 is disposed in an end surface 105 of the stem 104 at the first end 108.

A plurality of sealing rings or annular seals 112 extend radially outwardly from the stem 104. Each of the annular seals 112 extends circumferentially around the stem 104, and the annular seals are spaced apart along a length of the stem 104 between the first and second ends 108, 110. In use, an outer or circumferential edge 114 of each of the annular seals 112 contacts the wall of the air/water cylinder to form a seal. Furthermore, the grip or resistance provided by the annular seals on the wall of the air/water cylinder retains the sealing plug 102 within the air/water cylinder during use.

The sealing plug 102 further comprises a pair of apertures or through-holes 116, 118 in the stem 104. The apertures 116, 118 are aligned with each other and are disposed diametrically opposite each other. The apertures 116, 118 form a transverse passage 120 that extends across the stem 104. The transverse passage 120 intersects the longitudinal passage 106 to permit fluid flow between the longitudinal passage 106 and the transverse passage 120.

In this embodiment an axis of the transverse passage 120 is generally perpendicular to an axis of the longitudinal passage 106; however, in other embodiments the apertures 116, 118 may not be aligned in this way. Importantly, the apertures 116, 118 permit a flow of fluid from the longitudinal passage 106 to a region external to the stem 104.

A diameter of each of the apertures 116, 118 is preferably smaller than a diameter of the longitudinal passage 106.

The second end 110 of the tubular stem 104 is closed by a cover portion or head portion 122. The head portion 122 preferably includes a flange 124 or edge region that extends radially outwardly from the second end 110 of the tubular stem 104.

In this embodiment the cleaning adaptor 100 also comprises a sealing cap 130 for engagement with the suction port of the endoscope. The sealing cap 130 comprises a central stopper 132 including a plug portion 134 and a sealing surface 136 for engagement with the suction cylinder. In this embodiment the sealing cap 130 further comprises an annular retainer 138 that is configured to engage with the collar surrounding the opening of the suction cylinder. In this example the annular retainer 138 includes an undercut or recessed annular groove 140.

The annular retainer 138 extends around the central stopper 132 and a cover portion 142 of the sealing cap 130 connects the annular retainer 138 and the central stopper 132. The cover portion 142 provides an upper surface 144 of the sealing cap 130. A connecting portion 146 extends between and connects the head portion 122 of the sealing plug 102 and the cover portion 142 of the sealing cap 130. In this embodiment a lower surface 148 of the connecting portion 146 is continuous with a lower surface 150 of the flange 124 and an upper surface 152 of the connecting portion 146 is continuous with both an upper surface 154 of the head portion 122 and the upper surface 144 of the cover portion 142.

A grip element or grip portion 156 projects from the upper surface 144 of the cover portion 142. In this embodiment, the grip element 156 has a fin shape. The grip element 156 has two opposing grip surfaces 158. Grip features 160 in the form of elongate protrusions or ridges are provided on each of the grip surfaces 158 to assist a user in gripping the grip element 156. in this embodiment, the grip ridges 160 extend generally parallel to the upper surface 144 of the cover portion 142. It will be understood that, in other embodiments, the grip features 160 may have any suitable form to assist in gripping the grip element 156.

It is advantageous if the cleaning adapter 100 of the present invention is disposable or single use. Accordingly, in preferred embodiments, the cleaning adapter 100 includes a single use feature that prevents subsequent re-use of the adapter 100. The single use feature may comprise a part of the cleaning adapter that ruptures or breaks when the cleaning adapter is removed from the endoscope after use. The single use feature may comprise a part of the cleaning adapter that is disabled upon first use of the adapter such that the adapter cannot be reinserted into the air/water port of a second endoscope or cannot otherwise be reused.

In this embodiment, the cleaning adapter 100 includes a weakened portion 162 proximate the second end 110 of the stem 104 of the sealing plug 102. The weakened portion 162 is disposed between the head portion 122 and the annular seals 112.

As shown most clearly in FIGS. 8 and 13, a notch or indent 164 is provided in an external surface of the tubular stem 104 at the second end 110. In this way a part of a wall of the tubular stem 104 at the second end 110 has a smaller thickness than the rest of the tubular stem 104. This notch 164 or region of reduced thickness does not extend around the full circumference of the stem 104 and is disposed on a side of the tubular stem 104 closest to the sealing cap 130. The weakened portion 162 of the stem 104 is designed to rupture when a force is applied to the cleaning adaptor 100 to remove the adaptor 100 from the air/water port of an endoscope.

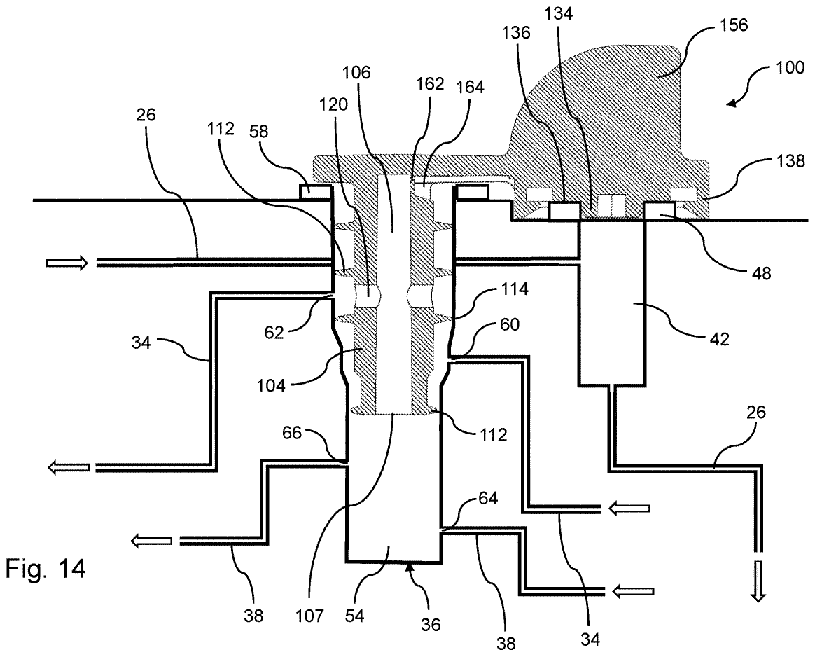

Use of the cleaning adaptor 100 will now be described with particular reference to FIG. 14.

After use of an endoscope during a medical procedure the air/water valve and the suction valve are removed to allow pre-cleaning of the endoscope. The cleaning adaptor 100 of the present invention is then engaged with the air/water port 36 and the suction port 30 of the control section of the endoscope.

The sealing plug 102 is pushed into the air/water port 36 so that the tubular stem 104 extends into the air/water cylinder 54 and a lower surface of the flange 124 of the head portion 122 preferably contacts the collar 58 surrounding the opening 56 of the air/water port 36.

The sealing cap 130 is engaged with the suction port 30 by inserting the plug portion 134 into the suction cylinder 42 so that the sealing surface 136 preferably contacts the collar 48 surrounding the opening 44 of the suction port 30. In some embodiments the annular retainer 138 may engage with a circumferential lip or undercut edge of the collar to retain the sealing cap 130 in engagement with the suction port 30.

With the sealing plug 102 inserted fully into the air/water cylinder 54, the annular seals 112 contact and seal against the internal surface of the wall of the air/water cylinder 54. A first one of the annular seals 112a is disposed at the first end 108 of the stem 104. The length of the stem 104 is such that the first end 108 of the stem 104 and the first seal 112a is disposed between the air inlet 60 and the water inlet 64 and between the air outlet 62 and the water outlet 66.

A second one of the annular seals 112b is disposed proximate the apertures 116, 118 of the transverse passage 120, between the apertures 116, 118 and the first annular seal 112a. In use, this second annular seal 112b is located such that the air inlet 60 is disposed between the first annular seal 112a and the second annular seal 112b.

A third one of the annular seals 112c is disposed proximate the apertures 116, 118 of the transverse passage 120, between the apertures 116, 118 and the second end 110 of the stem 104. In use, this third annular seal 112c is located such that the air outlet 62 is disposed between the second annular seal 112b and the third annular seal 112c. It will be appreciated that the apertures 116, 118 of the transverse passage 120 are also disposed between the second annular seal 112b and the third annular seal 112c. In this way a fluid flow path is formed between the transverse passage 120 and the air outlet 62.

In this embodiment a fourth one of the annular seals 112d is disposed proximate the second end 110 of the stem 104, between the third annular seal 112c and the second end 110. This fourth annular seal 112d therefore provides an additional seal between the transverse passage 120 and the second end 110 of the stem 104 to ensure that no fluid escapes through the opening 56 of the air/water port 36 in use. With the cleaning adaptor 100 fully engaged with the air/water port 36 and the suction port 30, a liquid is then introduced into the first section of the water channel 38. The liquid may be water, but preferably the liquid is a detergent solution.

The liquid enters the air/water cylinder 54 through the water inlet 64. As there are no seals between the water inlet 64 and the water outlet 66, the liquid is free to flow out of the water outlet 66 and into the second section of the water channel 38.

The liquid is also able to flow through the opening 107 in the end surface 105 of the stem 104 and into the passage 106. The liquid flows through the longitudinal passage 106, into the transverse passage 120, and out of the stem 104 through the apertures 116, 118. As described above, the apertures 116, 118 and the air outlet 62 are both disposed between the second and third annular seals 112b, 112c such that the liquid is able to flow through the air outlet 62 and into the second section of the air channel 34.

In this way, water or detergent solution may be flushed through the second section of both the air and water channels 34, 38 at the same time.

The sealing cap 130 of the cleaning adaptor 100 seals the opening 44 of the suction cylinder 42 so that fluid may also be flushed through the suction/biopsy channel 26 in a separate operation or method step. Preferably the suction/biopsy channel 26 is flushed with water or a detergent solution.

Once flushing of the air/water channels 34, 38 and suction/biopsy channel 26 has been completed, the cleaning adaptor 100 is removed from the air/water port 36 and suction port 30 of the endoscope. To do this a user preferably pulls on the grip element 156 to disengage the sealing cap 130 from the collar 48 around the opening 44 of the suction port 30. The user can then continue to apply a pulling force to the grip element 156 to pull the sealing plug 102 out of the air/water cylinder 54. It will be appreciated that a reasonable force must be applied to the sealing plug 102 to overcome the frictional force between each of the annular seals 112 and the internal surface of the wall of the air/water cylinder 54.

As a user pulls upwardly (in a direction away from the control section of the endoscope) on the grip element 156, this force is transferred to the head portion 122 of the sealing plug 102 via the cover portion 142 and connecting portion 146. The magnitude and direction of the force required to be applied to the head portion 122 to pull the stem 104 out of the air/water cylinder 54 is sufficient to break or rupture the weakened portion 162 of the stem 104. In this embodiment, the tensile force applied to the reduced thickness region 162 of the stem 104 is sufficient to tear the material in this region of the stem 104.

Once the weakened portion 162 of the stem 104 has been ruptured it will be appreciated that a fluid flow path is then formed from the longitudinal passage 106, through the ruptured portion and to an area external to the stem 104 between the head portion 122 and the annular seals 112. If the sealing plug 102 of the cleaning adaptor 100 is subsequently inserted into the air/water cylinder 54 of another endoscope, this fluid flow path through the ruptured portion will mean that the air/water port 36 can no longer be pressurised which prevents liquid being drawn up into the air/water cylinder 54.

Even if liquid is able to be drawn into the air/water cylinder 54, water or detergent is able to flow through the ruptured portion and out of the air/water cylinder 54 through the opening 56. As the resistance to fluid flow out of the air/water cylinder 54 through the opening 56 is less than the resistance to fluid flow along the air and water channels 34, 38 (due to the small diameter of each of the air and water channels), liquid will preferentially flow out of the air/water cylinder 54 through the opening 56 and not along the air and water channels 34, 38. Subsequent re-use of the cleaning adaptor 100 is, therefore, prevented.

Although in the above embodiment the cleaning adaptor 100 included both a sealing plug 102 for insertion into the air/water cylinder 54 of an endoscope and a sealing cap 130 for sealing the suction port 30 of an endoscope, in other embodiments the cleaning adaptor may only include a sealing plug and not a sealing cap. As discussed above the air/water channel and the suction channel are flushed in separate procedures. Accordingly, the cleaning adaptor may only include a sealing plug for use during flushing of the air/water channels.

In these embodiments, a grip element or grip portion may protrude from an upper surface of the head portion of the sealing plug. The grip element may be gripped by a user to both push or insert the stem of the sealing plug into the air/water cylinder and pull the stem of the sealing plug out of the air/water cylinder.

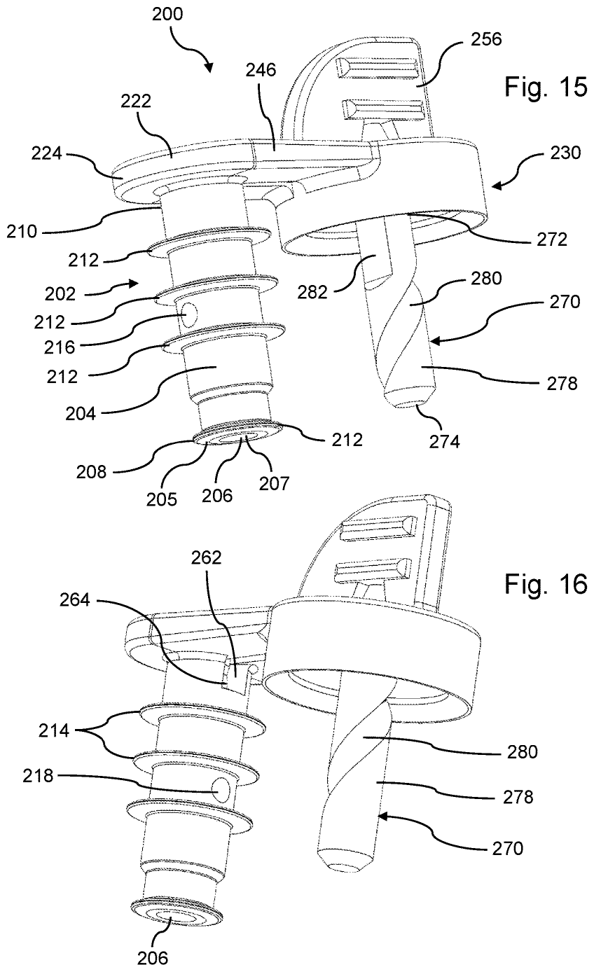

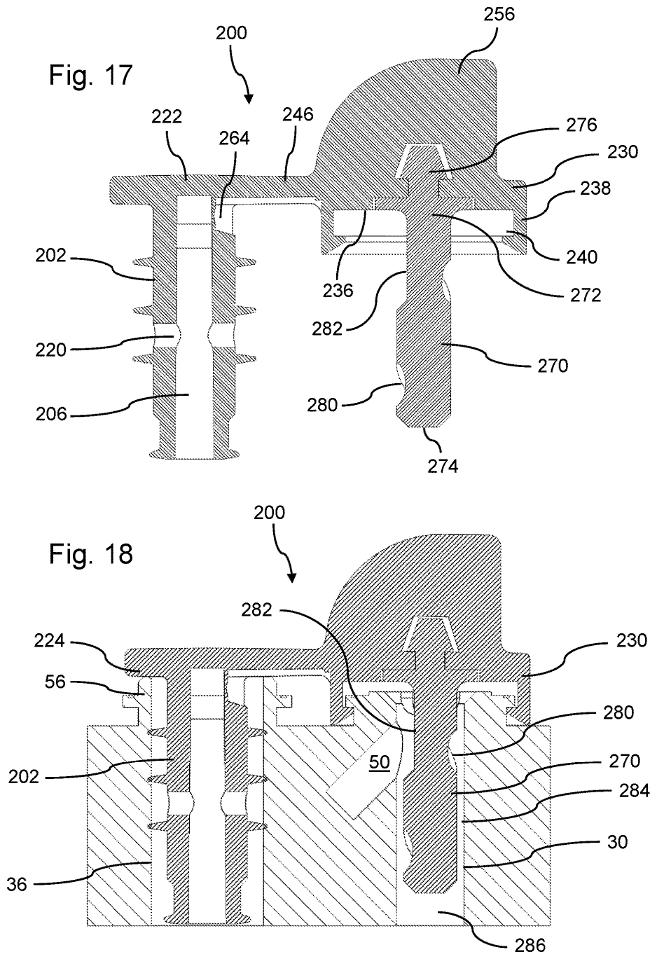

FIGS. 15 to 17 illustrate a cleaning adaptor 200 according to a second preferred embodiment of the present invention. The cleaning adaptor 200 comprises a sealing plug 202 for insertion into the air/water cylinder of the air/water port of the endoscope, a sealing cap 230 for engagement with and sealing of the suction port of the endoscope, and a flow modifying stem 270 extending from the sealing cap 230.

The sealing plug 202 of this embodiment is substantially identical to the sealing plug 102 of the first embodiment. The sealing plug 202 comprises an elongate tubular stem 204 having a passage or bore 206. The stem 204 extends between a first, lower or distal end 208 and a second, upper end 210. The passage 206 extends longitudinally through the stem 204 between the first and second ends 208, 210. An opening 207 at the end of the passage 206 is disposed in an end surface 205 of the stem 204 at the first end 208.

A plurality of sealing rings or annular seals 212 extend radially outwardly from the stem 204. Each of the annular seals 212 extends circumferentially around the stem 204, and the annular seals are spaced apart along a length of the stem 204 between the first and second ends 208, 210. In use, an outer or circumferential edge 214 of each of the annular seals 212 contacts the wall of the air/water cylinder to form a seal. Furthermore, the grip or resistance provided by the annular seals on the wall of the air/water cylinder retains the sealing plug 202 within the air/water cylinder during use.

The sealing plug 202 further comprises a pair of apertures or through-holes 216, 218 in the stem 204. The apertures 216, 218 are aligned with each other and are disposed diametrically opposite each other. The apertures 216, 218 form a transverse passage 220 that extends across the stem 204. The transverse passage 220 intersects the longitudinal passage 206 to permit fluid flow between the longitudinal passage 106 and the transverse passage 220.

In this embodiment an axis of the transverse passage 220 is generally perpendicular to an axis of the longitudinal passage 206; however, in other embodiments the apertures 216, 218 may not be aligned in this way. Importantly, the apertures 216, 218 permit a flow of fluid from the longitudinal passage 206 to a region external to the stem 204.

A diameter of each of the apertures 216, 218 is preferably smaller than a diameter of the longitudinal passage 206.

The second end 210 of the tubular stem 204 is closed by a cover portion or head portion 222. The head portion 222 preferably includes a flange 224 or edge region that extends radially outwardly from the second end 210 of the tubular stem 204.

In this embodiment the sealing cap 230 comprises a sealing surface 236 for engagement with the suction cylinder, and an annular retainer 238 that is configured to engage with the collar surrounding the opening of the suction cylinder. In this example the annular retainer 238 includes an undercut or recessed annular groove 240.

The flow modifying stem 270 is elongate and extends between first and second ends 272, 274. In this embodiment the flow modifying stem 270 is a separate component that is joined to the sealing cap 230. As such, the flow modifying stem 270 comprises a connector portion 276 at the first end 272 of the flow modifying stem 270. In this embodiment the connector portion 276 comprises a plug that engages with an aperture in the sealing cap 230 to attach the flow modifying stem 270 to the sealing cap 230.

It will be appreciated that in other embodiments the flow modifying stem 270 may be integrally formed with the sealing cap 230 so that the cleaning adaptor 200 is a unitary element.

The flow modifying stem 270 extends substantially perpendicularly from the sealing surface 236 of the sealing cap 230. In this embodiment a longitudinal axis of the flow modifying stem 270 is substantially parallel to the axis of the longitudinal passage 206 of the sealing plug 202.

The flow modifying stem 270 has a generally cylindrical outer surface 278 including a spiral channel or helical groove 280 extending along the length of the flow modifying stem 270. In the illustrated embodiment the helical groove 280 extends through a single 360° turn over the length of the flow modifying stem 270. In other embodiments the helical groove 280 may turn through more than 360° along the length of the flow modifying stem 270.

The outer surface 278 of the flow modifying stem 270 further comprises, in a region adjacent the sealing surface 236, a planar deflection surface 282. The planar deflection surface 282 preferably extends for less than half of the length of the flow modifying stem 270. In this example the planar deflection surface 282 is formed by a surface of a cylindrical segment oriented parallel to the longitudinal axis of the flow modifying stem 270.

As mentioned above, the cleaning adaptor 200 may be a unitary, one-piece element. In other embodiments the flow modifying stem 270 may be manufactured as a separate component which is then attached to or joined to the sealing cap 230. The cleaning adaptor 200 preferably has no moving parts other than due to the inherent flexibility of some parts of the adaptor 200 so that the cleaning adaptor is simple and inexpensive to manufacture. In some embodiments it is envisaged that the flow modifying stem 270 may be rotatable. This rotation may be driven by a flow of fluid around the flow modifying stem 270 as described further below.

The cleaning adaptor 200 is preferably made from a suitable polymeric material, having a degree of elasticity or flexibility. The cleaning adaptor may be made from a single material, and in these embodiments is preferably made from silicone. In other embodiments the cleaning adaptor 200 may be made from two materials. For example, the sealing plug 202 and sealing cap 230 may be made from a first material, for example silicone, and the flow modifying stem 270 may be made from a second material, for example polypropylene. The material from which the flow modifying stem 270 is made may include an anti-bacterial active agent. The material from which the flow modifying stem 270 is made may include silver ions, which may have anti-bacterial properties. The cleaning adaptor may be injection moulded, in one or more parts.

A connecting portion 246 extends between and connects the head portion 222 of the sealing plug 202 and the sealing cap 230.

A grip element or grip portion 256 projects from an upper surface of the sealing cap 230. In this embodiment, the grip element 256 has a fin shape and is substantially identical to the grip element 156 of the first embodiment.

It is advantageous if the cleaning adapter 200 of the present invention is disposable or single use. Accordingly, in preferred embodiments, the cleaning adapter 200 includes a single use feature that prevents subsequent re-use of the adapter 200. In this embodiment, the cleaning adapter 200 includes a weakened portion 262 proximate the second end 210 of the stem 204 of the sealing plug 202 in the form of a notch or indent 264 provided in an external surface of the tubular stem 204. This notch or indent 264 is substantially the same as the notch or indent 164 of the first embodiment and will not be described further in relation to this embodiment.

Use of the cleaning adaptor 200 will now be described with particular reference to FIGS. 14 and 18.

As described above in relation to the first embodiment, the cleaning adaptor 200 is engaged with the air/water port 36 and the suction port 30 of the control section of the endoscope prior to flushing of the air/water channels 34, 38 and suction channel 26 with water or detergent solution.

The sealing plug 202 is pushed into the air/water port 36 so that the tubular stem 204 extends into the air/water cylinder 54 and a lower surface of the flange 224 of the head portion 222 preferably contacts the collar 58 surrounding the opening 56 of the air/water port 36. With the sealing plug 202 inserted fully into the air/water cylinder 54, the annular seals 212 contact and seal against the internal surface of the wall of the air/water cylinder 54. The annular seals 212 are disposed relative to the air inlet 60, water inlet 64, air outlet 62 and water outlet 66 as described above in relation to the first embodiment.

The sealing cap 230 is engaged with the suction port 30 so that the sealing surface 236 preferably contacts the collar 48 surrounding the opening 44 of the suction port 30. In some embodiments the annular retainer 238 may engage with a circumferential lip or undercut edge of the collar to retain the sealing cap 230 in engagement with the suction port 30. In this position, the flow modifying stem 270 extends into the suction cylinder 42. An external diameter of the flow modifying stem 270 is smaller than an internal diameter of the suction cylinder 42 such that there is a cylindrical tubular gap 284 between the outer surface 278 of the flow modifying stem 270 and an inner surface of the suction cylinder 42. There is also a gap 286 between the second end 274 of the flow modifying stem 270 and the suction outlet 52 in base of the suction cylinder 42. Furthermore, the planar deflection surface 282 of the flow modifying stem 270 is disposed opposite the suction inlet 50.

With the cleaning adaptor 200 fully engaged with the air/water port 36 and the suction port 30, a liquid is then introduced into the first section of the water channel 38. The liquid may be water, but preferably the liquid is a detergent solution. The liquid enters and exits the air/water cylinder 54 as described above in relation to the first embodiment so that water or detergent solution may be flushed through the second section of both the air and water channels 34, 38 at the same time.

To flush the suction/biopsy channel 26 with a liquid (preferably water or a detergent solution) the liquid is drawn up through the second section of the suction/biopsy channel 26 and into the suction cylinder 42 through the suction inlet 50. As the liquid enters the suction cylinder 42 at least a part of the volume of the liquid strikes the planer deflection surface 282 and is deflected upwardly towards the opening 44 of the suction port 30 and the sealing surface 236. The liquid then flows towards the suction outlet 52 through the cylindrical tubular gap 284 between the outer surface 278 of the flow modifying stem 270 and the inner surface of the suction cylinder 42. The helical groove 280 in the outer surface 278 causes the liquid to flow around the flow modifying stem 270 thereby creating a vortex flow. This more turbulent flow in the suction cylinder 42 increases the ability of the liquid to clean or scour the inner surfaces of the suction cylinder 42. The turbulence of the flow of the liquid may be increased further by providing a textured outer surface 278 of the flow modifying stem 270, including surfaces of the helical groove 280. The liquid exits the suction cylinder through the suction outlet 52.

While in the above embodiment the flow modifying stem included a helical groove in an outer surface, in other embodiments the stem may not include such a groove. The presence of a cylindrical stem within the suction cylinder may be sufficient to create more turbulent fluid flow within the suction cylinder and/or to force the flow of fluid in the suction cylinder generally towards or against the inner surfaces of the suction cylinder. In other embodiments the outer surface of the generally cylindrical stem may be textured to provide a more turbulent fluid flow within the suction cylinder. In further embodiments the flow modifying stem may comprise fins or other protrusions extending radially outwardly from a central rod or shaft. The fins or protrusions may have a helical arrangement.

Although in this embodiment the sealing cap 230 and flow modifying stem 270 are joined or connected to the sealing plug 202, in other embodiments the sealing cap 230 and flow modifying stem 270 may be parts of a suction port plug that is separate from the sealing plug 202. Each of the separate and independent sealing plug and suction port plug may include a grip element so that each of the sealing plug and suction port plug can be easily removed from its respective port 36, 30.

In embodiments in which the sealing cap 230 and flow modifying stem 270 are parts of a suction port plug, the suction port plug preferably includes a single use feature to prevent the suction port plug being used more than once. The single use feature may comprise, for example, a weakened region of the sealing cap that tears or ruptures when a force is applied to the sealing cap to remove it from the opening of the suction port.

Although in the above embodiments the grip element projected from the upper surface of the cover portion or the head portion, in other embodiments the grip element or grip portion may be provided by a tab that extends from an edge of the cover portion or the head portion. The grip element may extend in a substantially radial direction from a circumferential edge of the cover portion or the head portion. In embodiments in which the tab extends from the cover portion of the sealing cap, the tab preferably extends in a direction generally away from the sealing plug.

Other modifications and variations not explicitly disclosed above may also be contemplated without departing from the scope of the invention as defined in the appended claims.

1. A cleaning adaptor for an air/water port of an endoscope, the cleaning adaptor being a unitary element comprising a sealing plug and a sealing cap connected to the sealing plug, the sealing plug having:
a tubular stem extending between first and second ends and including a longitudinal passage, the longitudinal passage being open at the first end of said stem;
a head portion closing the longitudinal passage at the second end of said stem and the head portion including a flange extending radially outwardly from the tubular stem;
at least three annular seals extending radially outwardly from the tubular stem and being spaced apart along a length of the tubular stem; and
a transverse passage extending through the tubular stem and providing a fluid flow path between the longitudinal passage and an aperture in an outer surface of the tubular stem, the aperture being disposed between two of the at least three annular seals, and
the sealing cap further comprising a plug portion for insertion into a suction cylinder of an endoscope and a cover portion including a sealing surface for contacting a collar surrounding an opening of said suction cylinder.

2. The cleaning adaptor according to claim 1, in which a first seal of the at least three annular seals is disposed at or proximate the first end of the stem, a second seal and a third seal of the at least three annular seals are disposed between the first seal and the second end of the stem, and the aperture is disposed between the second and third seals.

3. The cleaning adaptor according to claim 1, further comprising a weakened portion proximate the second end of the stem, the weakened portion being configured to rupture when a predetermined tensile force is applied to the weakened portion.

4. The cleaning adaptor according to claim 3, in which the weakened portion is disposed between the head portion and the annular seals.

5. The cleaning adaptor according to claim 3, in which the weakened portion comprises a notch or indent in an external surface of the tubular stem.

6. The cleaning adaptor according to claim 1, in which the transverse passage extends fully across the tubular stem and terminates at a first aperture on a first side of the stem and a second aperture on an opposite, second side of the stem.

7. (canceled)

8. The cleaning adaptor according to claim 1, in which the sealing cap further comprises an annular retainer including an annular groove configured to engage with said collar surrounding said opening of said suction cylinder.

9. The cleaning adaptor according to claim 1, in which the sealing plug is connected to the sealing cap by a flexible connecting portion extending between the head portion and the cover portion.

10. The cleaning adaptor according to claim 1, further comprising a grip element protruding from the head portion or the cover portion, the grip element configured to be gripped by a user of the cleaning adaptor.

11. (canceled)

12. The cleaning adaptor according to claim 1, wherein the unitary element is made from a resilient polymeric material.

13. An assembly comprising:
an endoscope having an air/water port connected to air and water channels and including an air/water cylinder having an air inlet, an air outlet, a water inlet and a water outlet, and a suction port including a suction cylinder having an opening surrounded by a collar; and
a cleaning adaptor in the form of a unitary element comprising a sealing plug and a sealing cap connected to the sealing plug, the sealing plug having:
a tubular stem extending between first and second ends and including a longitudinal passage, the longitudinal passage being open at the first end of said stem;
a head portion closing the longitudinal passage at the second end of said stem and the head portion including a flange extending radially outwardly from the tubular stem;
at least three annular seals extending radially outwardly from the tubular stem and being spaced apart along a length of the tubular stem; and
a transverse passage extending through the tubular stem and providing a fluid flow path between the longitudinal passage and an aperture in an outer surface of the tubular stem, the aperture being disposed between two of the at least three annular seals, and
the sealing cap comprising a plug portion for insertion into a suction cylinder of an endoscope and a cover portion including a sealing surface for contacting a collar surrounding an opening of said suction cylinder,
the sealing plug of the cleaning adaptor being disposed in the air/water cylinder such that the water inlet, air outlet and water outlet are in fluid communication with the longitudinal passage so that fluid is able to flow from the water inlet to both the air outlet and the water outlet, and at least two of the annular seals block fluid flow between the air inlet and any of the water inlet, the air outlet and the water outlet, and
the sealing cap engaged with the suction port such that the sealing surface of the cover portion is in contact with the collar to seal the suction cylinder.

14. The assembly according to claim 13, in which,
the first end of the stem and a first one of the annular seals is disposed between the air inlet and the water inlet and between the air outlet and the water outlet;
the air inlet is disposed between said first annular seal and a second one of the annular seals; and
the air outlet and the aperture is disposed between said second annular seal and a third one of the annular seals.

15. (canceled)

16. A method of cleaning air and water channels of an endoscope using a cleaning adaptor in the form of a unitary element comprising a sealing plug and a sealing cap connected to the sealing plug, the sealing plug having:
a tubular stem extending between first and second ends and including a longitudinal passage, the longitudinal passage being open at the first end of said stem;
a head portion closing the longitudinal passage at the second end of said stem and the head portion including a flange extending radially outwardly from the tubular stem;
at least three annular seals extending radially outwardly from the tubular stem and being spaced apart along a length of the tubular stem; and
a transverse passage extending through the tubular stem and providing a fluid flow path between the longitudinal passage and an aperture in an outer surface of the tubular stem, the aperture being disposed between two of the at least three annular seals, and
the sealing cap comprising a plug portion for insertion into a suction cylinder of an endoscope and a cover portion including a sealing surface for contacting a collar surrounding an opening of said suction cylinder,
the endoscope having an air/water port connected to the air and water channels and including an air/water cylinder having an air inlet, an air outlet, a water inlet and a water outlet, and a suction port connected to a suction channel, and the method comprising:
inserting a sealing plug of the cleaning adaptor into the air/water port wherein the tubular stem extends into the air/water cylinder and the air inlet is disposed between two annular seals such that there is no path for fluid flow between the air inlet and any of the water inlet, air outlet and water outlet, and wherein the water inlet, air outlet and water outlet are in fluid communication with the longitudinal passage;
engaging the sealing cap with a collar around an opening of the suction port to seal the suction port;
providing a flow of liquid to the air/water cylinder through the water inlet, the longitudinal passage and the transverse passage forming a fluid flow path allowing the liquid entering the air/water cylinder to flow out of both the air outlet and the water outlet simultaneously; and
flushing water or a detergent solution through the suction channel.

17. The method according to claim 16, in which the first end of the stem and a first one of the annular seals is disposed between the air inlet and the water inlet and between the air outlet and the water outlet, the air inlet is disposed between said first annular seal and a second one of the annular seals, and the air outlet and the aperture is disposed between said second annular seal and a third one of the annular seals.

18. The method according to claim 16, in which the endoscope comprises an air channel in fluid communication with both the air inlet and a container holding a volume of liquid, and a water channel in fluid communication with the water inlet and the container, and the method comprises providing a flow of air in the air channel to pressurize the container and force the liquid in the container to flow through the water channel to the water inlet to provide the flow of liquid to the air/water cylinder.

19. The method according to claim 16, in which the liquid is water or a detergent solution.

20. (canceled)

21. The method according to claim 16, in which the cleaning adaptor comprises a grip element protruding from the head portion configured to be gripped by a user of the cleaning adaptor and the method comprises gripping the grip element and applying a force to the cleaning adaptor to pull the sealing plug out of the air/water cylinder.

22. The method according to claim 16, in which the cleaning adaptor comprises a grip element protruding from the cover portion configured to be gripped by a user of the cleaning adaptor, and the method comprises gripping the grip element and applying a force to the cleaning adaptor to disengage the sealing cap from the collar of the suction port and to pull the sealing plug out of the air/water cylinder.

23. The method according to claim 21, in which the cleaning adaptor comprises a weakened portion proximate the second end of the stem, the weakened portion being configured to rupture when a predetermined tensile force is applied to the weakened portion, and the method comprises breaking or rupturing the weakened portion by applying a tensile force to the weakened portion while applying a force to the cleaning adaptor to pull the sealing plug out of the air/water cylinder.

24. The method according to claim 22, in which the cleaning adaptor comprises a weakened portion proximate the second end of the stem, the weakened portion being configured to rupture when a predetermined tensile force is applied to the weakened portion, and the method comprises breaking or rupturing the weakened portion by applying a tensile force to the weakened portion while applying a force to the cleaning adaptor to pull the sealing plug out of the air/water cylinder.
